(19) United States
(12) Patent Application Publication
(10) Pub. No.: US 2009/0131417 A1
(43) Pub. Date: May 21, 2009

(54) SUBSTITUTED PYRIDYL AMIDE COMPOUNDS AS MODULATORS OF THE HISTAMINE H3 RECEPTOR

(76) Inventors: Michael A. Letavic, San Diego, CA (US); Kiev S. Ly, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/272,355

(22) Filed: Nov. 17, 2008

Related U.S. Application Data
(60) Provisional application No. 60/989,244, filed on Nov. 20, 2007.

(51) Int. Cl. A61K 31/5513; C07D 401/06; A61K 31/497; C07D 401/14; C07D 405/14; A61P 25/00

(52) U.S. Cl. 514/218; 540/575; 514/253.12; 544/365

(57) ABSTRACT

Certain substituted pyridyl amide compounds are histamine H₃ receptor modulators useful in the treatment of histamine H₃ receptor-mediated diseases.

### CROSS REFERENCE TO RELATED APPLICATIONS

This application claims the benefit of U. S. Provisional Application 60/989,244, filed on Nov. 20, 2007, which is incorporated by reference herein in its entirety.

### FIELD OF THE INVENTION

The present invention relates to certain substituted pyridyl amide compounds, pharmaceutical compositions containing them, and methods of using them for the treatment of disease states, disorders, and conditions mediated by the histamine H₃ receptor.

### BACKGROUND OF THE INVENTION

The histamine H₃ receptor was first described as a presynaptic autoreceptor in the central nervous system (CNS) (Arrang, J.-M. et al. Nature 1983, 302, 832-837) controlling the synthesis and release of histamine. The histamine H₃ receptor is primarily expressed in the mammalian central nervous system (CNS), with some minimal expression in peripheral tissues such as vascular smooth muscle.

Thus, several indications for histamine H₃ antagonists and inverse agonists have been proposed based on animal pharmacology and other experiments with known histamine H₃ antagonists (e.g. thioperamide). (See: Krause et al. and Phillips et al. in “The Histamine H₃ Receptor-A Target for New Drugs”, Leurs, R. and Timmerman, H., (Eds.), Elsevier, 1998, pp. 175-196 and 197-222; Morisset, S. et al. Nature 2000, 408, 860-864.) These include conditions such as cognitive disorders, sleep disorders, psychiatric disorders, and other disorders.

For example, histamine H₃ antagonists have been shown to have pharmacological activity relevant to several key symptoms of depression, including sleep disorders (e.g. sleep disturbances, fatigue, and lethargy) and cognitive difficulties (e.g. memory and concentration impairment), as described above. For reviews, see: Bonaventure, P. et al. Biochem. Pharm. 2007, 73, 1084-1096; Letavic, M. A. et al. Prog. Med. Chem. 1996, 44, 181-206. There remains a need for potent histamine H₃ receptor modulators with desirable pharmaceutical properties.

Various literature publications describe small-molecule histamine H₃ receptor inhibitors: PCT Intl. Appl. Publ. WO 2005/040144 (diazepanyl derivatives); U.S. Pat. Appl. Publ. US 2007/219240 (N-substituted-azacyclylamines); U.S. Pat. Appl. Publ. US 2007/0167435 (phenoxypiperidines); U.S. Pat. Appl. Publ. US 2006/0178375 (heteroaryloxy nitrogen-containing derivatives); U.S. Pat. Appl. Publ. US 2006/0052597 (aryloxyalkylamine derivatives); U.S. Pat. Appl. Publ. US 2005/222151 (non-imidazole heterocyclic compounds; Attorney Docket No. PRD2208); U.S. Pat. Appl. Publ. US 2004/0019039 (substituted piperazines and diazepines); U.S. patent application Ser. No. 11/753,607 (Attorney Docket No. PRD2678); and U.S. patent application Ser. No. 11/766,144 (Attorney Docket No. PRD2686).

Substituted pyridines have been reported as angiogenic agents in U.S. Pat. Appl. Publ. US 2004/0014744 and as herbicides in U.S. Pat. No. 5,384,305 and U.S. Pat. No. 6,339,045.

### SUMMARY OF THE INVENTION

Certain substituted pyridyl amide derivatives have now been found to have histamine H₃ receptor modulating activity. Thus, the invention is directed to the general and preferred embodiments defined, respectively, by the independent and dependent claims appended hereto, which are incorporated by reference herein.

In one general aspect the invention relates to a compound of the following Formula (I):

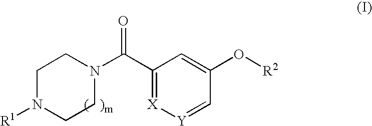

wherein
R¹ is —C₁₋₅alkyl or a saturated cycloalkyl group;
m is 1 or 2;
R² is an unsubstituted —C₂₋₄alkyl group, or a phenyl, a 6-membered monocyclic heteroaryl, a cycloalkyl, or a heterocycloalkyl ring, each ring unsubstituted or substituted with one or two R^{a} substituents;
   where each R^{a} substituent is independently halo, —C₁₋₄alkyl, acetyl, —CN, —CONR^{b}R^{c}, —OH, —OC₁₋₄alkyl, —SC₁₋₄alkyl, or —NO₂;
   where R^{b} and R^{c} are each independently —H or —C₁₋₄alkyl; and
one of X and Y is N and the other is CH;
or a pharmaceutically acceptable salt, a pharmaceutically acceptable prodrug, or a pharmaceutically active metabolite thereof.

In a further general aspect, the invention relates to pharmaceutical compositions each comprising: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

In another general aspect, the invention is directed to a method of treating a subject suffering from or diagnosed with a disease, disorder, or medical condition mediated by histamine H₃ receptor activity, comprising administering to the subject in need of such treatment an effective amount of a compound of Formula (I), or a pharmaceutically acceptable salt, pharmaceutically acceptable prodrug, or pharmaceutically active metabolite thereof.

In certain preferred embodiments of the inventive method, the disease, disorder, or medical condition is selected from: cognitive disorders, sleep disorders, psychiatric disorders, and other disorders.

Additional embodiments, features, and advantages of the invention will be apparent from the following detailed description and through practice of the invention.

### DETAILED DESCRIPTION

The invention may be more fully appreciated by reference to the following description, including the following glossary of terms and the concluding examples. For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a straight- or branched-chain alkyl group having from 1 to 12 carbon atoms in the chain. Examples of alkyl groups include methyl (Me, which also may be structurally depicted by a bond “/”), ethyl (Et), n-propyl, isopropyl (iPr), butyl (Bu or n-Bu), isobutyl (iBu), sec-butyl, tert-butyl (t-Bu), pentyl, isopentyl, tert-pentyl, hexyl, isohexyl, and groups that in light of the ordinary skill in the art and the teachings provided herein would be considered equivalent to any one of the foregoing examples.

The term “cycloalkyl” refers to a saturated or partially saturated, monocyclic carbocycle having from 3 to 10 ring atoms per carbocycle. Illustrative examples of cycloalkyl groups include the following entities, in the form of properly bonded moieties:

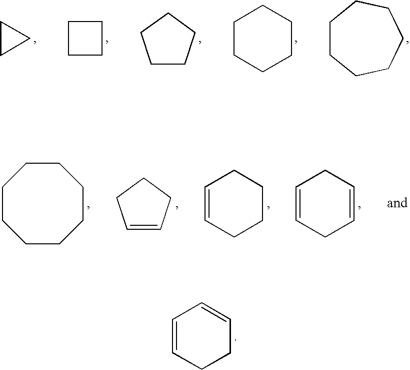

A “heterocycloalkyl” refers to a monocyclic ring structure that is saturated or partially saturated and has from 4 to 7 ring atoms per ring structure selected from carbon atoms and up to two heteroatoms selected from nitrogen, oxygen, and sulfur. The ring structure may optionally contain up to two oxo groups on sulfur ring members. Illustrative entities, in the form of properly bonded moieties, include:

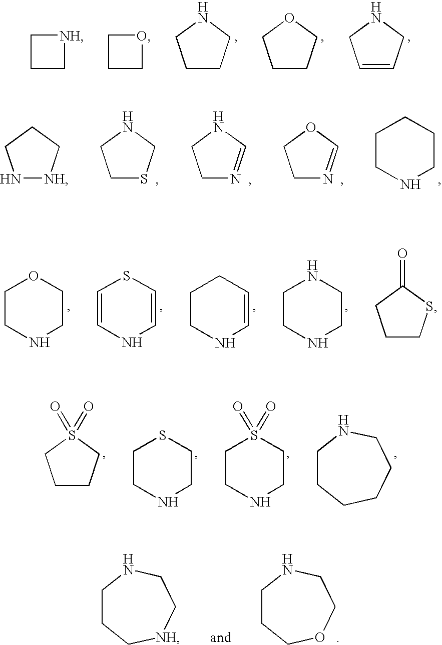

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

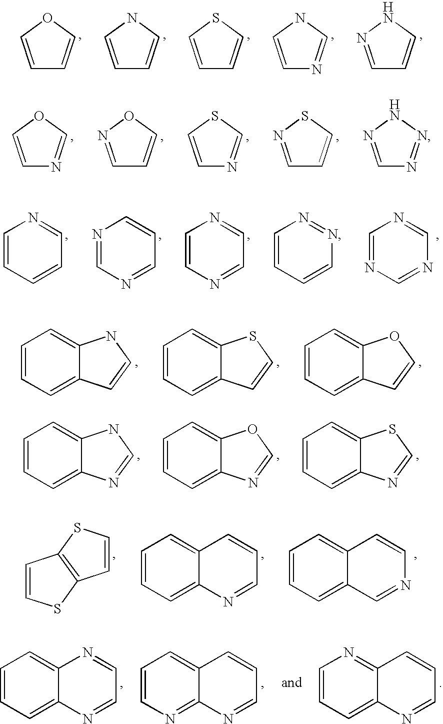

Those skilled in the art will recognize that the species of cycloalkyl, heterocycloalkyl, and heteroaryl groups listed or illustrated above are not exhaustive, and that additional species within the scope of these defined terms may also be selected.

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula.

In preferred embodiments of Formula (I), R¹ is isopropyl, cyclopropyl, cyclobutyl, or cyclopentyl. In other preferred embodiments, R¹ is cyclopropyl or cyclobutyl.

In some embodiments, m is 1. In other embodiments, m is 2.

In some embodiments, R² is phenyl, unsubstituted or substituted with a chloro, fluoro, methyl, cyano, methoxy, or methanesulfanyl group. In other embodiments, R² is phenyl, 4-fluorophenyl, 3-chlorophenyl, 3-cyanophenyl, or 3-methoxyphenyl. In other embodiments, R² is pyridinyl or pyrimidinyl. In other embodiments, R² is isopropyl. In still other embodiments, R² is cyclobutyl, cyclopentyl, cyclohexyl, tetrahydrofuranyl, tetrahydropyranyl, oxepanyl, tetrahydrothiophenyl, tetrahydrothiopyranyl, pyrrolidinyl, thiepanyl, piperidinyl, or azepanyl, each unsubstituted or substituted with methyl, ethyl, isopropyl, or acetyl.

In some embodiments, Y is N. In other embodiments, X is N.

In certain preferred embodiments, the compound of Formula (I) is selected from the group consisting of:

| Ex. | Chemical Name |
|---|---|
| 1 | (4-Cyclobutyl-[1,4]diazepan-1-yl)-[5-(4-fluoro-phenoxy)-pyridin-3-yl]-methanone; |
| 2 | [5-(3-Chloro-phenoxy)-pyridin-3-yl]-(4-cyclobutyl-[1,4]diazepan-1-yl)- |
| | methanone; |
| 3 | 3-[5-(4-Cyclobutyl-[1,4]diazepane-1-carbonyl)-pyridin-3-yloxy]- |
| | benzonitrile; |
| 4 | (4-Cyclobutyl-[1,4]diazepan-1-yl)-(5-phenoxy-pyridin-3-yl)-methanone; |
| 5 | (4-Cyclobutyl-[1,4]diazepan-1-yl)-[5-(3-methoxy-phenoxy)-pyridin-3-yl]- |
| | methanone; |
| 6 | (4-Cyclobutyl-[1,4]diazepan-1-yl)-[5-(3-fluoro-phenoxy)-pyridin-3-yl]- |
| | methanone; |
| 7 | (4-Cyclobutyl-[1,4]diazepan-1-yl)-(5-isopropoxy-pyridin-3-yl)-methanone; |
| 8 | (4-Cyclobutyl-[1,4]diazepan-1-yl)-[5-(4-fluoro-phenylsulfanyl)-pyridin-3- |
| | yl]-methanone; |
| 9 | (4-Cyclobutyl-[1,4]diazepan-1-yl)-(5-isopropylsulfanyl-pyridin-3-yl)- |
| | methanone; |
| 10 | (4-Cyclobutyl-[1,4]diazepan-1-yl)-[5-(pyridin-2-ylsulfanyl)-pyridin-3-yl]- |
| | methanone; |
| 11 | (4-Cyclobutyl-[1,4]diazepan-1-yl)-[5-(pyrimidin-2-ylsulfanyl)-pyridin-3-yl]- |
| | methanone; |
| 12 | (4-Cyclobutyl-[1,4]diazepan-1-yl)-[5-(pyridin-4-ylsulfanyl)-pyridin-3-yl]- |
| | methanone; |
| 13 | (4-Cyclobutyl-[1,4]diazepan-1-yl)-(4-phenoxy-pyridin-2-yl)-methanone; |
| 14 | (4-Cyclobutyl-[1,4]diazepan-1-yl)-[4-(4-fluoro-phenoxy)-pyridin-2-yl]- |
| | methanone; |
| 15 | (4-Cyclobutyl-[1,4]diazepan-1-yl)-[4-(4-fluoro-phenylsulfanyl)-pyridin-2- |
| | yl]-methanone; |
| 16 | (5-Cyclohexyloxy-pyridin-3-yl)-(4-isopropyl-[1,4]diazepan-1-yl)- |
| | methanone; |
| 17 | (4-Cyclopropyl-[1,4]diazepan-1-yl)-[5-(tetrahydro-furan-3-yloxy)-pyridin- |
| | 3-yl]-methanone; |
| 18 | (4-Cyclobutyl-[1,4]diazepan-1-yl)-[5-(tetrahydro-pyran-4-yloxy)-pyridin-3- |
| | yl]-methanone; |
| 19 | (4-Cyclopropyl-[1,4]diazepan-1-yl)-[5-(4-fluoro-phenoxy)-pyridin-3-yl]- |
| | methanone; |
| 20 | [5-(4-Chloro-phenoxy)-pyridin-3-yl]-(4-cyclopropyl-[1,4]diazepan-1-yl)- |
| | methanone; |
| 21 | (4-Cyclopropyl-[1,4]diazepan-1-yl)-[5-(3-fluoro-phenoxy)-pyridin-3-yl]- |
| | methanone; |
| 22 | 3-[5-(4-Cyclopropyl-[1,4]diazepane-1-carbonyl)-pyridin-3-yloxy]- |
| | benzonitrile; and |
| 23 | (4-Cyclopropyl-piperazin-1-yl)-[5-(4-fluoro-phenoxy)-pyridin-3-yl]- |
| | methanone; |

and pharmaceutically acceptable salts thereof.

The invention includes also pharmaceutically acceptable salts of the compounds of Formula (I), preferably of those described above and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response.

A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl)esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of the compounds of Formula (I), which may also be used in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites of the present invention are useful as modulators of the histamine H₃ receptor in the methods of the invention. As such modulators, the compounds may act as antagonists, agonists, or inverse agonists. “Modulators” include both inhibitors and activators, where “inhibitors” refer to compounds that decrease, prevent, inactivate, desensitize or down-regulate histamine H₃ receptor expression or activity, and “activators” are compounds that increase, activate, facilitate, sensitize, or up-regulate histamine H₃ receptor expression or activity.

The term “treat” or “treating” as used herein is intended to refer to administration of an active agent or composition of the invention to a subject for the purpose of effecting a therapeutic or prophylactic benefit through modulation of histamine H₃ receptor activity. Treating includes reversing, ameliorating, alleviating, inhibiting the progress of, lessening the severity of, or preventing a disease, disorder, or condition, or one or more symptoms of such disease, disorder or condition mediated through modulation of histamine H₃ receptor activity. The term “subject” refers to a mammalian patient in need of such treatment, such as a human.

Accordingly, the invention relates to methods of using the compounds described herein to treat subjects diagnosed with or suffering from a disease, disorder, or condition mediated by histamine H₃ receptor activity, such as: cognitive disorders, sleep disorders, psychiatric disorders, and other disorders. Symptoms or disease states are intended to be included within the scope of “medical conditions, disorders, or diseases.”

Cognitive disorders include, for example, dementia, Alzheimer's disease (Panula, P. et al., Soc. Neurosci. Abstr. 1995, 21, 1977), cognitive dysfunction, mild cognitive impairment (pre-dementia), attention deficit hyperactivity disorders (ADHD), attention-deficit disorders, and learning and memory disorders (Barnes, J. C. et al., Soc. Neurosci. Abstr. 1993, 19, 1813). Learning and memory disorders include, for example, learning impairment, memory impairment, age-related cognitive decline, and memory loss. H₃ antagonists have been shown to improve memory in a variety of memory tests, including the elevated plus maze in mice (Miyazaki, S. et al. Life Sci. 1995, 57(23), 2137-2144), a two-trial place recognition task (Orsetti, M. et al. Behav. Brain Res. 2001, 124(2), 235-242), the passive avoidance test in mice (Miyazaki, S. et al. Meth. Find. Exp. Clin. Pharmacol. 1995, 17(10), 653-658) and the radial maze in rats (Chen, Z. Acta Pharmacol. Sin. 2000, 21(10), 905-910). Also, in the spontaneously hypertensive rat, an animal model for the learning impairments in attention-deficit disorders, H₃ antagonists were shown to improve memory (Fox, G. B. et al. Behav. Brain Res. 2002, 131(1-2), 151-161).

Sleep disorders include, for example, insomnia, disturbed sleep, narcolepsy (with or without associated cataplexy), cataplexy, disorders of sleep/wake homeostasis, idiopathic somnolence, excessive daytime sleepiness (EDS), circadian rhythm disorders, fatigue, lethargy, jet lag (phase delay), and REM-behavioral disorder. Fatigue and/or sleep impairment may be caused by or associated with various sources, such as, for example, sleep apnea, perimenopausal hormonal shifts, Parkinson's disease, multiple sclerosis (MS), depression, chemotherapy, or shift work schedules.

Psychiatric disorders include, for example, schizophrenia (Schlicker, E. and Marr, I., Naunyn-Schmiedeberg's Arch. Pharmacol. 1996, 353, 290-294), including cognitive deficits and negative symptoms associated with schizophrenia, bipolar disorders, manic disorders, depression (Lamberti, C. et al. Br. J. Pharmacol. 1998, 123(7), 1331-1336; Perez-Garcia, C. et al. Psychopharmacology 1999, 142(2), 215-220) (Also see: Stark, H. et al., Drugs Future 1996, 21(5), 507-520; and Leurs, R. et al., Prog. Drug Res. 1995, 45, 107-165 and references cited therein.), including bipolar depression, obsessive-compulsive disorder, and post-traumatic stress disorder.

Other disorders include, for example, motion sickness, vertigo (e.g. vertigo or benign postural vertigo), tinitus, epilepsy (Yokoyama, H. et al., Eur. J. Pharmacol. 1993, 234, 129-133), migraine, neurogenic inflammation, neuropathic pain, Down Syndrome, seizures, eating disorders (Machidori, H. et al., Brain Res. 1992, 590, 180-186), obesity, substance abuse disorders, movement disorders (e.g. restless legs syndrome), and eye-related disorders (e.g. macular degeneration and retinitis pigmentosis).

Particularly, as modulators of the histamine H₃ receptor, the compounds of the present invention are useful in the treatment or prevention of depression, disturbed sleep, narcolepsy, fatigue, lethargy, cognitive impairment, memory impairment, memory loss, learning impairment, attention-deficit disorders, and eating disorders.

In treatment methods according to the invention, an effective amount of at least one compound according to the invention is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the compounds of the present invention may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.01 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the compounds of the invention may be used in combination with additional active ingredients in the treatment of the above conditions. In an exemplary embodiment, additional active ingredients are those that are known or discovered to be effective in the treatment of conditions, disorders, or diseases mediated by histamine H₃ receptor activity or that are active against another target associated with the particular condition, disorder, or disease, such as H₁ receptor antagonists, H₂ receptor antagonists, H₄ receptor antagonists, topiramate, and neurotransmitter modulators such as serotonin-norepinephrine reuptake inhibitors, selective serotonin reuptake inhibitors (SSRIs), noradrenergic reuptake inhibitors, non-selective serotonin re-uptake inhibitors (NSSRIs), acetylcholinesterase inhibitors (such as tetrahydroaminoacridine, donepezil, rivastigmine, or galantamine), or modafinil. The combination may serve to increase efficacy (e.g., by including in the combination a compound potentiating the potency or effectiveness of a compound according to the invention), decrease one or more side effects, or decrease the required dose of the compound according to the invention.

More particularly, compounds of the invention in combination with modafinil are useful for the treatment of narcolepsy, excessive daytime sleepiness (EDS), Alzheimer's disease, depression, attention-deficit disorders, MS-related fatigue, post-anesthesia grogginess, cognitive impairment, schizophrenia, spasticity associated with cerebral palsy, age-related memory decline, idiopathic somnolence, or jet-lag. Preferably, the combination method employs doses of modafinil in the range of about 20 to 300 mg per dose.

In another embodiment, compounds of the invention in combination with topiramate are useful for the treatment of obesity. Preferably, the combination method employs doses of topiramate in the range of about 20 to 300 mg per dose.

The compounds of the invention are used, alone or in combination with one or more other active ingredients, to formulate pharmaceutical compositions of the invention. A pharmaceutical composition of the invention comprises: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a compound of the invention and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the compounds of the invention may be prepared using suitable pharmaceutical excipients and compounding techniques now or later known or available to those skilled in the art. The compositions may be administered in the inventive methods by oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the compounds may be formulated to yield a dosage of, e.g., from about 0.01 to about 100 mg/kg daily, or from about 0.05 to about 35 mg/kg daily, or from about 0.1 to about 10 mg/kg daily.

Oral tablets may include a compound according to the invention mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the invention may be mixed with a solid, semi-solid, or liquid diluent. Soft gelatin capsules may be prepared by mixing the compound of the invention with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds of this invention may also be administered by non-oral routes. For example, the compositions may be formulated for rectal administration as a suppository. For parenteral use, including intravenous, intramuscular, intraperitoneal, or subcutaneous routes, the compounds of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the invention may utilize a patch formulation to affect transdermal delivery.

Compounds of the invention may alternatively be administered in methods of this invention by inhalation, via the nasal or oral routes, e.g., in a spray formulation also containing a suitable carrier.

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent.

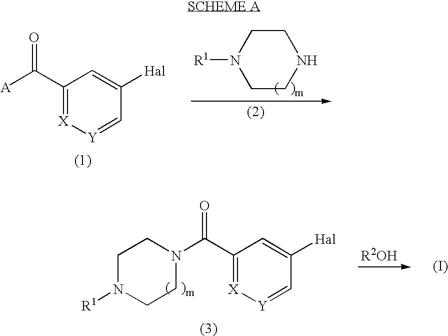

In some embodiments, compounds of Formula (I) are prepared as shown in Scheme A. Halo-pyridines (1), where Hal is bromo or chloro and A is OH, Cl, Br, or —OC(O)C₁₋₄alkyl, are commercially available or prepared using methods known to one skilled in the art. Amide coupling of such acids (1) with amines (2), in the presence of coupling agents known to one skilled in the art, provides amides (3). Alternatively, acid halides (1) (where A is Cl or Br) or mixed anhydrides (1) (where A is —OC(O)C₁₋₄alkyl) may be reacted with amines (2) in the presence of a suitable base such as aq. NaOH, aq. KOH, Et₃N, iPr₂NEt, pyridine, or a mixture thereof, in a solvent such as CH₂Cl₂, dichloroethane (DCE), N,N-dimethylformamide (DMF), toluene, isopropyl acetate, or a mixture thereof, to form amides (3). One skilled in the art will recognize that the R¹ substitutent may be carried through the sequence as a suitable protecting group (such as a tert-butylcarbamoyl, or Boc, group), and installed at a later point in the sequence by, for example, reductive amination protocols.

Displacement of the Hal substituent is accomplished by reaction with reagents R²OH, in the presence of a suitable base such as NaOH, KOH, K₂CO₃, Na₂CO₃, Cs₂CO₃, NaH, or a mixture thereof, in a polar solvent such as DMF, ethylene glycol dimethyl ether (DME), N,N-dimethylacetamide (DMA), dimethylsulfoxide (DMSO), acetonitrile, or a mixture thereof, at a temperature between room temperature and the reflux temperature of the solvent, or subject to microwave irradiation at a temperature up to about 250° C., to provide compounds of Formula (I).

Those skilled in the art will recognize that several of the chemical transformations described above may be performed in a different order than that depicted in the above Schemes.

Compounds of Formula (I) may be converted to their corresponding salts using methods known to those skilled in the art. For example, amines of Formula (I) may be treated with trifluoroacetic acid (TFA), HCl, maleic acid, or citric acid in a solvent such as diethyl ether (Et₂O), CH₂Cl₂, tetrahydrofuran (THF), or methanol (MeOH) to provide the corresponding salt forms.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following examples are provided to further illustrate the invention and various preferred embodiments.

### EXAMPLES

### Chemistry:

In obtaining the compounds described in the examples below and the corresponding analytical data, the following experimental and analytical protocols were followed unless otherwise indicated.

Unless otherwise stated, reaction mixtures were magnetically stirred at room temperature (rt). Where solutions were “dried,” they were generally dried over a drying agent such as Na₂SO₄ or MgSO₄. Where mixtures, solutions, and extracts were “concentrated”, they were typically concentrated on a rotary evaporator under reduced pressure. Reactions under microwave irradiation conditions were carried out in a Biotage Initiator or CEM Discover instrument.

Normal-phase flash column chromatography (FCC) was performed on silica gel (SiO₂) using prepackaged cartridges.

Preparative reverse-phase high performance liquid chromatography (HPLC) was performed on a Gilson HPLC with an Xterra Prep RP₁₈ (5 μm, 30×100 mm) column, and a gradient of 10 to 99% acetonitrile/water (20 mM NH₄OH) over 12 to 18 min, and a flow rate of 30 mL/min.

Mass spectra (MS) were obtained on an Agilent series 1100 MSD using electrospray ionization (ESI) in positive mode unless otherwise indicated. Calculated (calcd.) mass corresponds to the exact mass.

Nuclear magnetic resonance (NMR) spectra were obtained on Bruker model DRX spectrometers. The format of the ¹H NMR data below is: chemical shift in ppm downfield of the tetramethylsilane reference (multiplicity, coupling constant J in Hz, integration).

Chemical names were generated using ChemDraw Version 6.0.2 (CambridgeSoft, Cambridge, Mass.).

### Example 1:

### (4-Cyclobutyl-[1,4]diazepan-1-yl)-[5-(4-fluoro-phenoxy)-pyridin-3-yl]-methanone

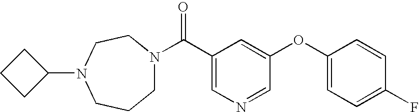

Step A: (5-Bromo-pyridin-3-yl)-(4-cyclobutyl-[1,4]diazepan-1-yl)-methanone. To a solution of 5-bromonicotinic acid (73.3 mmol) in CH₂Cl₂ (0.5 M) was added DMF (catalytic, ˜100 μL) followed by oxalyl chloride (29.7 mmol). The reaction mixture was allowed to stir at rt for 1 h. The mixture was concentrated, diluted with CH₂Cl₂, and concentrated again. The resulting residue was diluted with CH₂Cl₂ (0.5 M), cooled to 0° C., and treated with Et₃N (74.2 mmol) followed by 1-cyclobutyl-[1,4]diazepane dihydrochloride (17.8 mmol). The reaction mixture was allowed to warm to rt and was stirred for 18 h. The mixture was diluted with CH₂Cl₂ and washed with satd. aq. NaHCO₃ (2×). The organic layer was dried and concentrated to provide the title compound (4.74 g, 94%). MS (ESI): mass calcd. for C₁₅H₂₀BrN₃O, 337.08; m/z found, 388.0 [M+H]⁺. ¹H NMR (500 MHz, CDCl₃): δ 8.75-8.69 (m, 1H), 8.60-8.56 (m, 1H), 7.96-7.86 (m, 1H), 3.84-3.70 (m, 2H), 3.52-3.44 (m, 2H), 3.02-2.76 (m, 1H), 2.67-2.61 (m, 1H), 2.55-2.49 (m, 1H), 2.49-2.43 (m, 2H), 2.13-1.91 (m, 3H), 1.92-1.52 (m, 7H).

Step B. A mixture of (5-bromo-pyridin-3-yl)-(4-cyclobutyl-[1,4]diazepan-1-yl)-methanone (0.30 mmol), 4 fluorophenol (0.59 mmol), and Cs₂CO₃ (0.59 mmol) in DMA (0.2 M) was heated in the microwave reactor for 2 h at 200° C. The mixture was treated with additional Cs₂CO₃ (0.3 mmol), and heated for an additional 2 h at 200° C. The reaction mixture was diluted with Et₂O, washed sequentially with water, 1 M NaOH, and satd. aq. NaCl, dried, and concentrated. The residue was purified by preparative reverse-phase HPLC. The desired fractions were combined, basified with 1 M NaOH, extracted with CH₂Cl₂, dried, and concentrated to provide the title compound (17.02 mmol, 57%). MS (ESI): mass calcd. for C₂₁H₂₄FN₃O₂, 369.19; m/z found, 370.3 [M+H]⁺. ¹H NMR (400 MHz, CDCl₃): δ 8.38 (dd, J=12.0, 2.2 Hz, 2H), 7.25 (dd, J=2.8, 1.8 Hz, 1H), 7.14-6.97 (m, 4H), 3.80-3.70 (m, 2H), 3.50-3.39 (m, 2H), 2.96-2.77 (m, 1H), 2.64-2.57 (m, 1H), 2.53-2.46 (m, 1H), 2.46-2.38 (m, 2H), 2.14-1.90 (m, 3H), 1.90-1.52 (m, 5H).

The compounds in Examples 2-12 were prepared using methods analogous to those described for Example 1.

### Example 2

### [5-(3-Chloro-phenoxy)-pyridin-3-yl]-(4-cyclobutyl-[1,4]diazepan-1-yl)-methanone

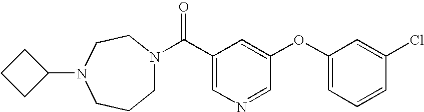

MS (ESI): mass calcd. for C₂₁H₂₄ClN₃O₂, 385.16; m/z found, 386.2 [M+H]⁺. ¹H NMR (500 MHz, CDCl₃): δ 8.48-8.40 (m, 2H), 7.37-7.30 (m, 2H), 7.21-7.16 (m, 1H), 7.09-7.05 (m, 1H), 6.98-6.93 (m, 1H), 3.82-3.71 (m, 2H), 3.53-3.43 (m, 2H), 2.98-2.80 (m, 1H), 2.69-2.59 (m, 1H), 2.55-2.41 (m, 3H), 2.12-1.92 (m, 3H), 1.92-1.53 (m, 5H).

### Example 3

### 3-[5-(4-Cyclobutyl-[1,4]diazepane-1-carbonyl)-pyridin-3-yloxy]-benzonitrile

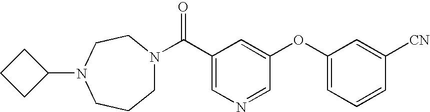

MS (ESI): mass calcd. for C₂₂H₂₄N₄O₂, 376.19; m/z found, 377.3 [M+H]⁺. ¹H NMR (500 MHz, CDCl₃): δ 8.50-8.47 (m, 1H), 8.47-8.43 (m, 1H), 7.55-7.45 (m, 2H), 7.40-7.36 (m, 1H), 7.35-7.32 (m, 1H), 7.32-7.28 (m, 1H), 3.83-3.71 (m, 2H), 3.54-3.46 (m, 2H), 2.98-2.81 (m, 1H), 2.67-2.59 (m, 1H), 2.54-2.41 (m, 3H), 2.13-1.93 (m, 3H), 1.90-1.54 (m, 5H).

### Example 4

### (4-Cyclobutyl-[1,4]diazepan-1-yl)-(5-phenoxy-pyridin-3-yl)-methanone

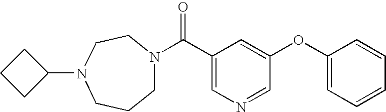

MS (ESI): mass calcd. for C₂₁H₂₅N₃O₂, 351.19; m/z found, 352.3 [M+H]⁺. ¹H NMR (400 MHz, CDCl₃): δ 8.42 (d, J=2.7 Hz, 1H), 8.37 (d, J=1.7 Hz, 1H), 7.45-7.34 (m, 2H), 7.29 (dd, J=2.7, 1.8 Hz, 1H), 7.24-7.16 (m, 1H), 7.09-7.01 (m, 2H), 3.80-3.68 (m, 2H), 3.51-3.40 (m, 2H), 2.96-2.78 (m, 1H), 2.65-2.55 (m, 1H), 2.52-2.37 (m, 3H), 2.12-1.91 (m, 3H), 1.90-1.51 (m, 5H).

### Example 5

### (4-Cyclobutyl-[1,4]diazepan-1-yl)-[5-(3-methoxy-phenoxy)-pyridin-3-yl]-methanone

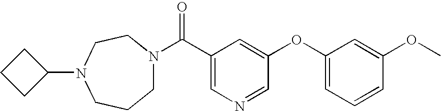

MS (ESI): mass calcd. for C₂₂H₂₇N₃O₃, 381.21; m/z found, 382.3 [M+H]⁺. ¹H NMR (500 MHz, CDCl₃): δ 8.45-8.43 (m, 1H), 8.40-8.37 (m, 1H), 7.33-7.27 (m, 2H), 6.78-6.74 (m, 1H), 6.66-6.61 (m, 2H), 3.83-3.80 (m, 3H), 3.79-3.74 (m, 2H), 3.51-3.43 (m, 2H), 2.97-2.79 (m, 1H), 2.65-2.59 (m, 1H), 2.53-2.48 (m, 1H), 2.47-2.41 (m, 2H), 2.11-1.91 (m, 3H), 1.90-1.55 (m, 5H).

### Example 6

### (4-Cyclobutyl-[1,4]diazepan-1-yl)-[5-(3-fluoro-phenoxy)-pyridin-3-yl]-methanone

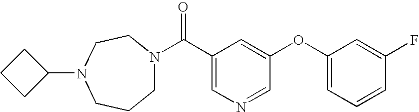

MS (ESI): mass calcd. for C₂₁H₂₄FN₃O₂, 369.19; m/z found, 370.3 [M+H]⁺. ¹H NMR (400 MHz, CDCl₃): δ 8.45-8.40 (m, 2H), 7.38-7.30 (m, 2H), 6.94-6.86 (m, 1H), 6.86-6.81 (m, 1H), 6.81-6.74 (m, 1H), 3.81-3.71 (m, 2H), 3.51-3.40 (m, 2H), 2.96-2.77 (m, 1H), 2.65-2.58 (m, 1H), 2.53-2.39 (m, 3H), 2.12-1.88 (m, 3H), 1.86-1.54 (m, 5H).

### Example 7

### (4-Cyclobutyl-[1,4]diazepan-1-yl)-(5-isopropoxy-pyridin-3-yl)-methanone

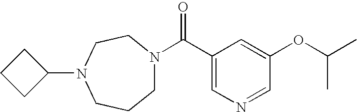

MS (ESI): mass calcd. for C₁₈H₂₇N₃O₂, 317.21; m/z found, 318.3 [M+H]⁺. ¹H NMR (500 MHz, CD₃OD): δ 8.79-8.72 (m, 1H), 8.62-8.56 (m, 1H), 8.15-8.08 (m, 1H), 3.86-3.68 (m, 2H), 3.54-3.43 (m, 2H), 3.34-3.27 (m, 7H), 3.04-2.91 (m, 1H), 2.73-2.63 (m, 1H), 2.61-2.42 (m, 3H), 2.16-2.00 (m, 2H), 1.99-1.78 (m, 4H), 1.77-1.58 (m, 2H).

### Example 8

### (4-Cyclobutyl-[1,4]diazepan-1-yl)-[5-(4-fluoro-phenylsulfanyl)-pyridin-3-yl]-methanone

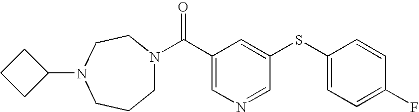

MS (ESI): mass calcd. for C₂₁H₂₄FN₃OS, 385.16; m/z found 386.2 [M+H]⁺. ¹H NMR (500 MHz, CDCl₃): δ 8.50-8.42 (m, 2H), 7.51-7.45 (m, 3H), 7.14-7.07 (m, 2H), 3.81-3.68 (m, 2H), 3.47-3.35 (m, 2H), 2.96-2.79 (m, 1H), 2.64-2.58 (m, 1H), 2.51-2.46 (m, 1H), 2.45-2.35 (m, 2H), 2.12-1.91 (m, 3H), 1.89-1.56 (m, 5H).

### Example 9

### (4-Cyclobutyl-[1,4]diazepan-1-yl)-(5-isopropylsulfanyl-pyridin-3-yl)-methanone

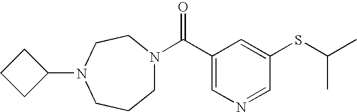

MS (ESI): mass calcd. for C₁₈H₂₇N₃OS, 333.19; m/z found 334.3 [M+H]⁺. ¹H NMR (500 MHz, CDCl₃): δ 8.64-8.60 (m, 1H), 8.51-8.48 (m, 1H), 7.76-7.72 (m, 1H), 3.83-3.74 (m, 2H), 3.52-3.38 (m, 3H), 2.98-2.82 (m, 1H), 2.69-2.61 (m, 1H), 2.55-2.50 (m, 1H), 2.49-2.42 (m, 2H), 2.12-1.59 (m, 8H), 1.35-1.31 (m, 6H).

### Example 10

### (4-Cyclobutyl-[1,4]diazepan-1-yl)-[5-(pyridin-2-ylsulfanyl)-pyridin-3-yl]-methanone

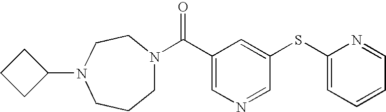

MS (ESI): mass calcd. for C₂₀H₂₄N₄OS, 368.17; m/z found 369.3 [M+H]⁺. ¹H NMR (500 MHz, CDCl₃): δ 8.80-8.75 (m, 1H), 8.68-8.64 (m, 1H), 8.43-8.38 (m, 1H), 7.96-7.93 (m, 1H), 7.60-7.54 (m, 1H), 7.17-7.13 (m, 1H), 7.13-7.08 (m, 1H), 3.83-3.75 (m, 2H), 3.57-3.48 (m, 2H), 2.98-2.83 (m, 1H), 2.67-2.60 (m, 1H), 2.54-2.50 (m, 1H), 2.49-2.42 (m, 2H), 2.12-1.93 (m, 3H), 1.90-1.56 (m, 5H).

### Example 11

### (4-Cyclobutyl-[1,4]diazepan-1-yl)-[5-(pyrimidin-2-ylsulfanyl)-pyridin-3-yl]-methanone

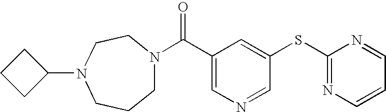

MS (ESI): mass calcd. for C₁₉H₂₃N₅OS, 369.16; m/z found 370.2 [M+H]⁺. ¹H NMR (500 MHz, CDCl₃): δ 8.85-8.81 (m, 1H), 8.73-8.69 (m, 1H), 8.53-8.46 (m, 2H), 8.03-7.99 (m, 1H), 7.09-7.01 (m, 1H), 3.85-3.73 (m, 2H), 3.60-3.50 (m, 2H), 2.98-2.80 (m, 1H), 2.68-2.61 (m, 1H), 2.56-2.51 (m, 1H), 2.50-2.41 (m, 2H), 2.13-1.94 (m, 3H), 1.91-1.53 (m, 5H).

### Example 12

### (4-Cyclobutyl-[1,4]diazepan-1-yl)-[5-(pyridin-4-ylsulfanyl)-pyridin-3-yl]-methanone

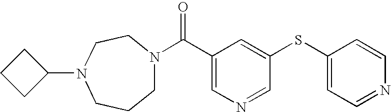

MS (ESI): mass calcd. for C₂₀H₂₄N₄OS, 368.17; m/z found 369.3 [M+H]⁺. ¹H NMR (500 MHz, CDCl₃): δ 8.77-8.67 (m, 2H), 8.46-8.39 (m, 2H), 7.92-7.86 (m, 1H), 7.05-6.99 (m, 2H), 3.82-3.72 (m, 2H), 3.52-3.43 (m, 2H), 2.99-2.78 (m, 1H), 2.66-2.60 (m, 1H), 2.54-2.49 (m, 1H), 2.48-2.42 (m, 2H), 2.12-1.91 (m, 3H), 1.89-1.53 (m, 5H)

### Example 13

### (4-Cyclobutyl-[1,4]diazepan-1-yl)-(4-phenoxy-pyridin-2-yl)-methanone

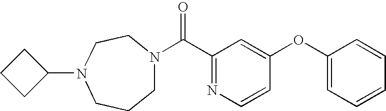

Step A: (4-Bromo-pyridin-2-yl)-(4-cyclobutyl-[1,4]diazepan-1-yl)-methanone. A mixture of 4-bromo-pyridine-2-carboxylic acid (9.9 mmol), 1-cyclobutyl-[1,4]diazepane dihydrochloride (11.9 mmol), bromotripyrrolidino-phosphonium hexafluorophosphate (11.9 mmol), and iPr₂NEt (19.6 mmol) in CH₂Cl₂ (0.5 M) was stirred at rt for 18 h. The mixture was diluted with 1 M NaOH and extracted with CH₂Cl₂. The organic layer was washed with satd. aq. NaCl, dried, and concentrated. The residue was purified by FCC (0.1% NH₄OH/MeOH in DCM) to give the title compound (1.94 g, 58%). MS (ESI): mass calcd. for C₁₅H₂₀BrN₃O, 338.08; m/z found, 388.2 [M+H]⁺. ¹H NMR (500 MHz, CDCl₃): δ 8.40-8.36 (m, 1H), 7.81-7.77 (m, 1H), 7.52-7.45 (m, 1H), 3.81-3.75 (m, 2H), 3.63-3.53 (m, 2H), 2.95-2.83 (m, 1H), 2.65-2.60 (m, 1H), 2.54-2.44 (m, 3H), 2.10-1.94 (m, 3H), 1.90-1.54 (m, 6H).

Step B. A mixture of (4-bromo-pyridin-2-yl)-(4-cyclobutyl-[1,4]diazepan-1-yl)-methanone (0.80 mmol) and Cs₂CO₃ (1.60 mmol) in DMA (0.2 M) was heated in the microwave reactor for 3 h at 200° C. The reaction mixture was diluted with Et₂O, washed sequentially with water, 1 M NaOH, and satd. aq. NaCl, dried, and concentrated. The residue was purified by preparative reverse-phase HPLC. The desired fractions were combined, basified with 1 M NaOH, and extracted with CH₂Cl₂. The organic layer was dried and concentrated to provide the title compound (0.31 mmol, 38%). MS (ESI): mass calcd. for C₂₁H₂₅N₃O₂, 351.19; m/z found 352.3 [M+H]⁺. ¹H NMR (500 MHz, CDCl₃): δ 8.49-8.36 (m, 1H), 7.48-7.41 (m, 2H), 7.31-7.25 (m, 1H), 7.16-7.08 (m, 3H), 6.90-6.84 (m, 1H), 3.82-3.73 (m, 2H), 3.63-3.59 (m, 1H), 3.59-3.54 (m, 1H), 2.97-2.83 (m, 1H), 2.67-2.59 (m, 1H), 2.54-2.45 (m, 3H), 2.12-1.92 (m, 3H), 1.91-1.76 (m, 3H), 1.73-1.57 (m, 2H).

The compounds in Examples 14-15 were prepared using methods analogous to those described for Example 13, with exceptions where noted.

### Example 14

### (4-Cyclobutyl-[1,4]diazepan-1-yl)-[4-(4-fluoro-phenoxy)-pyridin-2-yl]-methanone

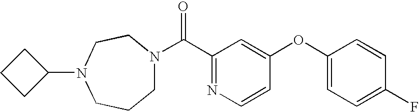

MS (ESI): mass calcd. for C₂₁H₂₄FN₃O₂, 369.19; m/z found, 370.3 [M+H]⁺. ¹H NMR (400 MHz, CDCl₃): δ 8.53-8.28 (m, 1H), 7.17-7.01 (m, 5H), 6.86-6.81 (m, 1H), 3.83-3.70 (m, 2H), 3.64-3.51 (m, 2H), 2.96-2.81 (m, 1H), 2.66-2.57 (m, 1H), 2.55-2.42 (m, 3H), 2.11-1.92 (m, 3H), 1.91-1.52 (m, 5H).

### Example 15

### (4-Cyclobutyl-[1,4]diazepan-1-yl)-[4-(4-fluoro-phenylsulfanyl)-pyridin-2-yl]-methanone

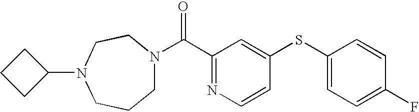

The reaction for Step B was performed at rt. MS (ESI): mass calcd. for C₂₁H₂₄FN₃OS, 385.16; m/z found 386.2 [M+H]⁺. ¹H NMR (500 MHz, CDCl₃): δ 8.33-8.28 (m, 1H), 7.60-7.54 (m, 2H), 7.21-7.14 (m, 3H), 6.93-6.89 (m, 1H), 3.81-3.71 (m, 2H), 3.59-3.55 (m, 1H), 3.54-3.49 (m, 1H), 2.96-2.82 (m, 1H), 2.65-2.58 (m, 1H), 2.53-2.42 (m, 3H), 2.11-1.93 (m, 3H), 1.90-1.55 (m, 5H).

The compounds in Examples 16-23 may be prepared using methods analogous to those described for the preceding examples.

### Example 16

### (5-Cyclohexyloxy-pyridin-3-yl)-(4-isopropyl-[1,4]diazepan-1-yl)-methanone

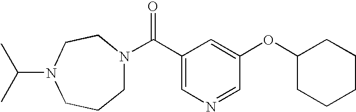

### Example 17

### (4-Cyclopropyl-[1,4]diazepan-1-yl)-[5-(tetrahydro-furan-3-yloxy)-pyridin-3-yl]-methanone

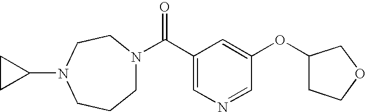

### Example 18

### (4-Cyclobutyl-[1,4]diazepan-1-yl)-[5-(tetrahydro-pyran-4-yloxy)-pyridin-3-yl]-methanone

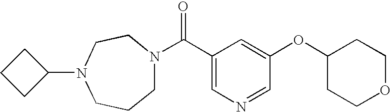

### Example 19

### (4-Cyclopropyl-[1,4]diazepan-1-yl)-[5-(4-fluoro-phenoxy)-pyridin-3-yl]-methanone

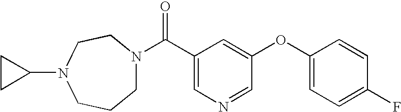

### Example 20

### [5-(4-Chloro-phenoxy)-pyridin-3-yl]-(4-cyclopropyl-[1,4]diazepan-1-yl)-methanone

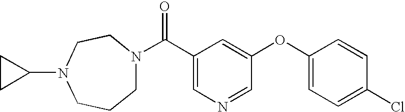

### Example 21

### (4-Cyclopropyl-[1,4]diazepan-1-yl)-[5-(3-fluoro-phenoxy)-pyridin-3-yl]-methanone

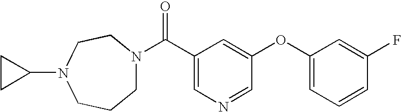

### Example 22

### 3-[5-(4-Cyclopropyl-[1,4]diazepane-1-carbonyl)-pyridin-3-yloxy]-benzonitrile

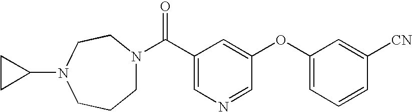

### Example 23

### (4-Cyclopropyl-piperazin-1-yl)-[5-(4-fluoro-phenoxy)-pyridin-3-yl]-methanone

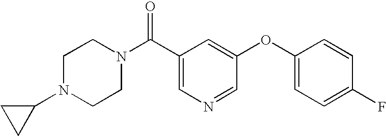

### Biological Methods:

### H₃ Receptor Binding (Human)

Binding of compounds to the cloned human H₃ receptors, stably expressed in SK-N-MC cells, was performed as described by Barbier, A. J. et al. (Br. J. Pharmacol. 2004, 143(5), 649-661). Data for compounds tested in this assay are presented in Table 1, as an average of results obtained.

TABLE 1

| | Human H₃ |
|---|---|
| Ex. | Kᵢ (nM) |
| 1 | 3.6 |
| 2 | 3.2 |
| 3 | 1.9 |
| 4 | 3.4 |
| 5 | 3.4 |
| 6 | 1.0 |
| 7 | 11 |
| 8 | 2.9 |
| 9 | 3.0 |
| 10 | 6.6 |
| 11 | 18 |
| 12 | 1.8 |
| 13 | 11 |
| 14 | 17 |
| 15 | 12 |

### H₃ Receptor Binding (Rat)

A rat brain without cerebellum (Zivic Laboratories Inc., Pittsburgh, Pa.) was homogenized in 50 mM Tris-HCl/5 mM EDTA and centrifuged at 1,000 rpm for 5 min. The supernatant was removed and recentrifuged at 15,000 rpm for 30 min. Pellets were rehomogenized in 50 mM Tris/5 mM EDTA (pH 7.4). Membranes were incubated with 0.8 nM N—[³H]-α-methylhistamine plus/minus test compounds for 60 min at 25° C. and harvested by rapid filtration over GF/C glass fiber filters (pretreated with 0.3% polyethylenimine) followed by four washes with buffer. Nonspecific binding was defined in the presence of 100 μM histamine. Inhibitory concentration (responsible for 50% inhibition of maximal effect, IC₅₀) values were determined by a single site curve-fitting program (GraphPad, San Diego, Calif.) and converted to Kᵢ values based on a N—[³H]-α-methylhistamine dissociation constant (K_{d}) of 0.8 nM. The following results were obtained: Example 2, 28 nM; Example 6, 50 nM (tested as the corresponding TFA salt).

### Cyclic AMP Accumulation

Sublines of SK-N-MC cells were created that expressed a reporter construct and either the human or rat H₃ receptor. The pA₂ values were obtained as described by Barbier et al. (2004). Data for compounds tested in this assay are presented in Table 2. Example 1 was tested as the corresponding TFA salt.

TABLE 2

| Ex. | Human pA₂ | Rat pA₂ |
|---|---|---|
| 1 | 8.87 | 7.54 |
| 3 | 9.02 | 8.21 |
| 6 | 8.62 | 7.69 |
| 8 | 8.40 | 7.36 |

What is claimed is:

1. A compound of Formula (I): 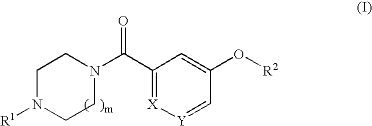
wherein
R¹ is —C₁₋₅alkyl or a saturated cycloalkyl group;
m is 1 or 2;
R² is an unsubstituted —C₂₋₄alkyl group, or a phenyl, a 6-membered monocyclic heteroaryl, a cycloalkyl, or a heterocycloalkyl ring, each ring unsubstituted or substituted with one or two R^{a} substituents;
where each R^{a} substituent is independently halo, —C₁₋₄alkyl, acetyl, —CN, —CONR^{b}R^{c}, —OH, —OC₁₋₄alkyl, —SC₁₋₄alkyl, or —NO₂;
where R^{b} and R^{c} are each independently —H or —C₁₋₄alkyl; and
one of X and Y is N and the other is CH;
or a pharmaceutically acceptable salt, a pharmaceutically acceptable prodrug, or a pharmaceutically active metabolite thereof.

2. A compound as defined in claim 1, wherein R¹ is isopropyl, cyclopropyl, cyclobutyl, or cyclopentyl.

3. A compound as defined in claim 1, wherein R¹ is cyclopropyl or cyclobutyl.

4. A compound as defined in claim 1, wherein m is 1.

5. A compound as defined in claim 1, wherein m is 2.

6. A compound as defined in claim 1, wherein R² is phenyl, unsubstituted or substituted with a chloro, fluoro, methyl, cyano, methoxy, or methanesulfanyl group.

7. A compound as defined in claim 1, wherein R² is phenyl, 4-fluorophenyl, 3-chlorophenyl, 3-cyanophenyl, or 3-methoxyphenyl.

8. A compound as defined in claim 1, wherein R² is pyridinyl or pyrimidinyl.

9. A compound as defined in claim 1, wherein R² is isopropyl.

10. A compound as defined in claim 1, wherein R² is cyclobutyl, cyclopentyl, cyclohexyl, tetrahydrofuranyl, tetrahydropyranyl, oxepanyl, tetrahydrothiophenyl, tetrahydrothiopyranyl, pyrrolidinyl, thiepanyl, piperidinyl, or azepanyl, each unsubstituted or substituted with methyl, ethyl, isopropyl, or acetyl.

11. A compound as defined in claim 1, wherein Y is N.

12. A compound as defined in claim 1, wherein X is N.

13. A compound selected from the group consisting of:
(4-Cyclobutyl-[1,4]diazepan-1-yl)-[5-(4-fluoro-phenoxy)-pyridin-3-yl]-methanone;
[5-(3-Chloro-phenoxy)-pyridin-3-yl]-(4-cyclobutyl-[1,4]diazepan-1-yl)-methanone;
3-[5-(4-Cyclobutyl-[1,4]diazepane-1-carbonyl)-pyridin-3-yloxy]-benzonitrile;
(4-Cyclobutyl-[1,4]diazepan-1-yl)-(5-phenoxy-pyridin-3-yl)-methanone;
(4-Cyclobutyl-[1,4]diazepan-1-yl)-[5-(3-methoxy-phenoxy)-pyridin-3-yl]-methanone;
(4-Cyclobutyl-[1,4]diazepan-1-yl)-[5-(3-fluoro-phenoxy)-pyridin-3-yl]-methanone;
(4-Cyclobutyl-[1,4]diazepan-1-yl)-(5-isopropoxy-pyridin-3-yl)-methanone;
(4-Cyclobutyl-[1,4]diazepan-1-yl)-[5-(4-fluoro-phenylsulfanyl)-pyridin-3-yl]-methanone;
(4-Cyclobutyl-[1,4]diazepan-1-yl)-(5-isopropylsulfanyl-pyridin-3-yl)-methanone;
(4-Cyclobutyl-[1,4]diazepan-1-yl)-[5-(pyridin-2-ylsulfanyl)-pyridin-3-yl]-methanone;
(4-Cyclobutyl-[1,4]diazepan-1-yl)-[5-(pyrimidin-2-ylsulfanyl)-pyridin-3-yl]-methanone;
(4-Cyclobutyl-[1,4]diazepan-1-yl)-[5-(pyridin-4-ylsulfanyl)-pyridin-3-yl]-methanone;
(4-Cyclobutyl-[1,4]diazepan-1-yl)-(4-phenoxy-pyridin-2-yl)-methanone;
(4-Cyclobutyl-[1,4]diazepan-1-yl)-[4-(4-fluoro-phenoxy)-pyridin-2-yl]-methanone;
(4-Cyclobutyl-[1,4]diazepan-1-yl)-[4-(4-fluoro-phenylsulfanyl)-pyridin-2-yl]-methanone;
(5-Cyclohexyloxy-pyridin-3-yl)-(4-isopropyl-[1,4]diazepan-1-yl)-methanone;
(4-Cyclopropyl-[1,4]diazepan-1-yl)-[5-(tetrahydro-furan-3-yloxy)-pyridin-3-yl]-methanone;
(4-Cyclobutyl-[1,4]diazepan-1-yl)-[5-(tetrahydro-pyran-4-yloxy)-pyridin-3-yl]-methanone;
(4-Cyclopropyl-[1,4]diazepan-1-yl)-[5-(4-fluoro-phenoxy)-pyridin-3-yl]-methanone;
[5-(4-Chloro-phenoxy)-pyridin-3-yl]-(4-cyclopropyl-[1,4]diazepan-1-yl)-methanone;
(4-Cyclopropyl-[1,4]diazepan-1-yl)-[5-(3-fluoro-phenoxy)-pyridin-3-yl]-methanone;
3-[5-(4-Cyclopropyl-[1,4]diazepane-1-carbonyl)-pyridin-3-yloxy]-benzonitrile; and
(4-Cyclopropyl-piperazin-1-yl)-[5-(4-fluoro-phenoxy)-pyridin-3-yl]-methanone;
and pharmaceutically acceptable salts thereof.

14. A pharmaceutical composition for treating a disease, disorder, or medical condition mediated by histamine H₃ receptor activity, comprising:
(a) an effective amount of a compound of Formula (I): 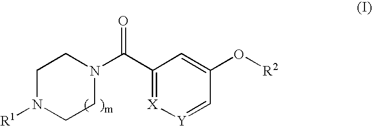
wherein
R¹ is —C₁₋₅alkyl or a saturated cycloalkyl group;
m is 1 or 2;
R² is an unsubstituted —C₂₋₄alkyl group, or a phenyl, a 6-membered monocyclic heteroaryl, a cycloalkyl, or a heterocycloalkyl ring, each ring unsubstituted or substituted with one or two R^{a} substituents;
where each R^{a} substituent is independently halo, —C₁₋₄alkyl, acetyl, —CN, —CONR^{b}R^{c}, —OH, —OC₁₋₄alkyl, —SC₁₋₄alkyl, or —NO₂;
where R^{b} and R^{c} are each independently —H or —C₁₋₄alkyl; and
one of X and Y is N and the other is CH;
or a pharmaceutically acceptable salt, pharmaceutically acceptable prodrug, or pharmaceutically active metabolite thereof; and
(b) a pharmaceutically acceptable excipient.

15. A method of treating a subject suffering from or diagnosed with a disease, disorder, or medical condition mediated by histamine H₃ receptor activity, comprising administering to the subject in need of such treatment an effective amount of a compound of Formula (I): 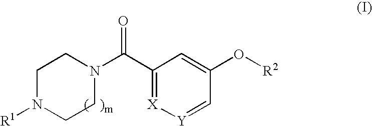
wherein
R¹ is —C₁₋₅alkyl or a saturated cycloalkyl group;
m is 1 or 2;
R² is an unsubstituted —C₂₋₄alkyl group, or a phenyl, a 6-membered monocyclic heteroaryl, a cycloalkyl, or a heterocycloalkyl ring, each ring unsubstituted or substituted with one or two R^{a} substituents;
where each R^{a} substituent is independently halo, —C₁₋₄alkyl, acetyl, —CN, —CONR^{b}R^{c}, —OH, —OC₁₋₄alkyl, —SC₁₋₄alkyl, or —NO₂;
where R^{b} and R^{c} are each independently —H or —C₁₋₄alkyl; and
one of X and Y is N and the other is CH;
or a pharmaceutically acceptable prodrug, or pharmaceutically active metabolite thereof.

16. The method according to claim 15, wherein the disease, disorder, or medical condition is selected from the group consisting of: cognitive disorders, sleep disorders, psychiatric disorders, and other disorders.

17. The method according to claim 15, wherein the disease, disorder, or medical condition is selected from the group consisting of: dementia, Alzheimer's disease, cognitive dysfunction, mild cognitive impairment, pre-dementia, attention deficit hyperactivity disorders, attention-deficit disorders, learning and memory disorders, learning impairment, memory impairment, age-related cognitive decline, and memory loss, insomnia, disturbed sleep, narcolepsy with or without associated cataplexy, cataplexy, disorders of sleep/wake homeostasis, idiopathic somnolence, excessive daytime sleepiness, circadian rhythm disorders, fatigue, lethargy, jet lag, REM-behavioral disorder, sleep apnea, perimenopausal hormonal shifts, Parkinson's disease, multiple sclerosis, depression, chemotherapy, shift work schedules, schizophrenia, bipolar disorders, manic disorders, depression, obsessive-compulsive disorder, post-traumatic stress disorder, motion sickness, vertigo, benign postural vertigo, tinitus, epilepsy, migraine, neurogenic inflammation, neuropathic pain, Down Syndrome, seizures, eating disorders, obesity, substance abuse disorders, movement disorders, restless legs syndrome, eye-related disorders, macular degeneration, and retinitis pigmentosis.

18. The method according to claim 15, wherein the disease, disorder, or medical condition is selected from the group consisting of: depression, disturbed sleep, fatigue, lethargy, cognitive impairment, memory impairment, memory loss, learning impairment, attention-deficit disorders, and eating disorders.
